(19) United States
(12) Patent Application Publication
(10) Pub. No.: US 2002/0000012 A1
(43) Pub. Date: Jan. 3, 2002

(54) Compositions and methods for dyeing keratin fibers, especially hair

(76) Inventors: Juergen Schmenger, Weiterstadt (DE); Wilhelm Abels, Simi Valley, CA (US); Manfred Schmitt, Heppenheim (DE)

Correspondence Address: STRIKER, STRIKER & STENBY, 103 East Neck Road, Huntington, NY, 11743, US

(21) Appl. No.: 09/833,975

(22) Filed: Apr. 12, 2001

(30) Foreign Application Priority Data
May 11, 2000 (DE) 100 23 028.8

(51) Int. Cl. A61K 7/13

(52) U.S. Cl. 8/405; 8/406

(57) ABSTRACT

Compositions for dyeing keratin fibers, especially human hair, contain oxidation dye pre-cursor compounds and/or at least one direct-dyeing dye compound and at least one nonionic, amphiphilic associative thickener in a suitable cosmetic carrier. Methods of dyeing hair with these dye compositions are also described.

### BACKGROUND OF THE INVENTION

1. Field of the Invention

The present invention relates to compositions for dyeing keratin fibers, especially human hair, containing direct-dyeing and/or oxidative dye compounds and certain associative thickeners, as well as to methods for dyeing keratin fibers, especially human hair, using the compositions.

2. Prior Art

Conventional dyeing preparations are usually in the form of aqueous, preferably thickened, solutions or emulsion and especially contain fatty alcohol and/or other oily components, emulsifiers and surfactants, and if necessary alcohol, in addition to dye compounds. Oxidiation dye compositions usually comprise two components (i) the dye carrier containing the dye compounds and (ii) the oxidizing preparation, which are mixed immediately prior to application on the hair to form a dyeing mixture, which is then applied to the hair. A higher or lower viscosity results from the mixing according to the respective viscosity and mixture ratio of each component. Good adherence of the dyeing mixture is obtained when the dyeing mixture has a high viscosity. Also the hairstyle often requires a higher viscosity for desired work, especially to be able to perform desired work with a special dyeing brush or accentuating brush and with special strand or foil techniques. However this requires a definite increase of the amount of thickener with the conventionally used thickener and leads to a definite increase of the cost of the preparation. Also uniform mixing of both components is difficult because of the high viscosity. However as an alternative in certain cases it is possible to increase the viscosity of the dyeing agent subsequently, but the color shade may not be changed by the viscosity adjustment.

A subsequent increase in the viscosity of the dyeing mixture has not been possible up to now without changing the color shade for either oxidative or non-oxidative dye compositions.

### SUMMARY OF THE INVENTION

It is an object of the present invention to provide improved dye compositions and methods for dyeing keratin fibers and methods of dyeing keratin fibers, in which the above-described disadvantages are reduced or eliminated.

It is a further object of the present invention to satisfy the great need for an economical thickener for the dye carrier, which guarantees good miscibility of the dye carrier with the oxidation agent preparation, which permits easy subsequent increase of the viscosity of the ready-to-apply hair dyeing mixture, as needed, and which provides a dyeing mixture including good adherence and dyeing properties.

For this purpose it has now been found that dyeing compositions containing certain nonionic amphiphilic associative thickeners fulfill the requirements for the dyeing mixture, especially regarding viscosity and adherence, and also regarding color shade properties, in an outstanding manner.

According to the invention the dye composition for dyeing keratin fibers, especially hair, comprises oxidation dye pre-cursor compounds and/or direct-dyeing dye compounds as well as at least one nonionic amphiphilic associative thickener in a suitable cosmetic carrier.

The nonionic amphiphilic associative thickener is present in the composition according to the invention in an amount of about 0.01 to 20 percent by weight, especially preferably of from about 0.1 to 10 percent by weight.

A polymer, which contains both hydrophilic and hydrophobic groups, is used as nonionic amphiphilic associative thickener. Associative thickeners are water-soluble polymers and have surfactant-like hydrophobic parts, which are in a position to associate both with themselves and also with other hydrophobic materials in a hydrophilic, especially aqueous medium. The medium is thickened or gels because of the resulting associative network. Typically associative thickeners are made by polymerization of polyethylene oxide pre-polymers and polycondensible materials with at least two functional groups, such as isocyanates. Monohydroxy compounds or dihydroxy compounds are built in with large aryl groups, alkyl groups or aryl/alkyl groups, in order to prepare the hydrophobic modifications. Hydrophobic modified polyalkylene glycols are preferred associative thickeners. The hydrophilic functional groups are preferably formed from hydrocarbon groups, especially alkyl groups, alkyl aryl groups or arylalkyl groups. Hydrophobic modified aminoplast polyether copolymers are particularly preferred as associative thickeners. WO 96/40815 describes the structure and manufacture of these latter compounds. Water-dispersible or water-soluble copolymers are described in WO 96/40815, which are the acid catalyzed reaction products of aminoplast monomers with at least two functional groups and alkylene polyethers with at least two functional groups as well as functional compounds with hydrophobic groups. Suitable aminoplasts are shown in FIG. 1 WO 96/40815. The glycoluril derivatives of formula X of WO 96/40815 are especially preferred. Suitable alkylpolyethers are shown in FIG. 2 of WO 96/40815. Preferred alkylene polymers are polyethyleneoxidediols. These compounds can have an ethoxylation degree of 20 to 500, preferably 50 to 350 and especially preferably from 100 to 250. The compounds of formula XIV of WO 96/40815 are suitable simple compounds with hydrophobic groups.

According to the invention suitable associative thickeners are preferably selected from the group consisting of polymers of the general formula (I):

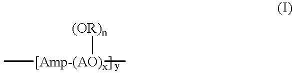

wherein Amp represents an aminoplast monomer or an aminoplast oligomer group or an aminoplast polymer; AO represents an alkylene oxide group, R represents hydrogen, a C₁- to C₄-alkyl group or a C₁- to C₄-acyl group and x and y are greater than 1.

The reaction products of acid catalyzed polycondensation of (a) glycolurils of the general formula (II) are especially preferred as the associative thickener,

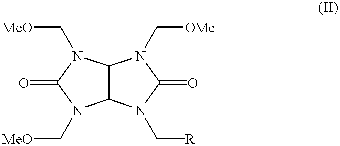

wherein R represents H or preferably OMe, with (b) polyethylene oxide diols having an ethoxylation degree of 20 to 500, preferably 50 to 350, especially preferably from 100 to 250, and, if necessary, with (c) an—if need be ethoxylated-hydrophobic alcohol or alkyphenol, a thiol, a carboxamide, a carbamate or a hydrophobic carboxylic acid, as described on pages 17 to 19 of WO 96/40815. 1,3,4,6-tetramethoxymethyl glycoluril (TMMG) is especially preferred as the glycouril used in the composition of the invention.

Those compounds, which are designated with the following INCI names, are especially preferred as the associative thickener: polyether-1, PEG-180/octoxynol-40/TMMG copolymer and PEG-180/Laureth-50/TMMG copolymer. These compounds are, for example, marketed by Süd-Chemie AG, München, Germany, under the trademark Pure-Thix® HH, HL, L, M, TX-1442, TX-1450, TX-1451, TX-1452 and TX-1499.

The dye composition according to the invention contains, preferably oxidation dye precursors. The dyed colors are produced by the oxidation dye precursors under the action of an oxidizing agent, such as hydrogen peroxide, or in the presence of oxygen.

The following developer substances and coupler substances are suitable as oxidation dye precursor compounds.

The suitable developer compounds include 1,4-diamino-benzene (p-phenylenediamine), 1,4-diamino-2-methyl-benzene (p-toluylenediamine), 1,4-diamino-2,6-dimethyl-benzene, 1,4-diamino-3,5-diethyl-benzene, 1,4-diamino-2,5-dimethyl-benzene, 1,4-diamino-2,3-dimethyl-benzene, 2-chloro-1,4-diaminobenzene, 1,4-diamino-2-(thiophen-2-yl)benzene, 1,4-diamino-2-(thio-phen-3-yl)benzene, 1,4-diamino-2-(pyridin-3-yl)benzene, 2,5-diaminobiphenyl, 1,4-diamino-2-methoxymethyl-benzene, 1,4-diamino-2-aminomethylbenzene, 1,4-diamino-2-hydroxymethyl-benzene, 1,4-diamino-2-(2-hydroxyethoxy)-benzene, 2-(2-(acetylamino)ethoxy)-1,4-diaminobenzene, 4-phenylamino-aniline, 4-dimethylamino-aniline, 4-diethylamino-aniline, 4-dipropylamino-aniline, 4-[ethyl(2-hydroxyethyl)amino]-aniline, 4-[di(2-hydroxyethyl)amino]-aniline, 4-[di(2-hydroxyethyl)amino]-2-methyl-aniline, 4-[(2-methoxyethyl)amino]-aniline, 4-[(3-hydroxypropyl)amino]-aniline, 4-[(2,3-dihydroxypropyl)amino-aniline, 1,4-diamino-2-(2-hydroxyethyl)-benzene, 1,4-diamino-2-(1-methylethyl)-benzene, 1,3-Bis[(4-aminophenyl)(2-hydroxyethyl)amino]-2-propanol, 1,4-Bis[(4-aminophenyl)amino]-butane, 1,8-Bis(2,5-diaminophenoxy)-3,6-dioxaoctane, 4-amino-phenol, 4-amino-3-methyl-phenol, 4-amino-3-(hydroxymethyl)-phenol, 4-amino-3-fluoro-phenol, 4-methylamino-phenol, 4-amino-2-(aminomethyl)-phenol, 4-amino-2-(hydroxymethyl)-phenol, 4-amino-2-fluoro-phenol, 4-amino-2-[(2-hydroxyethyl)-amino]methylphenol, 4-amino-2-methyl-phenol, 4-amino-2-(methoxymethyl)-phenol, 4-amino-2-(2-hydroxyethyl)-phenol, 5-amino-salicylic acid, 2,5-diamino-pyridine, 2,4,5,6-tetraamino-pyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chloro-phenyl)methyl]-4,5-diamino-1H-pyrazole, 4,5-diamino-1-methyl-1H-pyrazole, 2-aminophenol, 2-amino-6-methylphenol and 2-amino-5-methylphenol. The developer compounds may be used individually or together with each other in a mixture.

The suitable coupler substances include N-(3-dimethylamino-phenyl)-urea, 2,6-diamino-pyridine, 2-amino-4-[(2-hydroxyethyl)amino]anisole, 2,4-diamino-1-fluoro-5-methyl-benzene, 2,4-diamino-1-methoxy-5-methylbenzene, 2,4-diamino-1-ethoxy-5-methyl-benzene, 2,4-diamino-1-(2-hydroxyethoxy)-5-methylbenzene, 2,4-di[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxy-pyridine, 1,3-diaminobenzene, 2,4-diamino-1-(2-hydroxyethoxy)benzene, 1,3-diamino-4-(2,3-dihydroxypropoxy)benzene, 2,4-diamino-1,5-di(2-hydroxyethoxy)-benzene, 1-(2-aminoethoxy)-2,4-diaminobenzene, 2-amino-1-(2-hydroxyethoxy)-4-methylaminobenzene, 2,4-diaminophenoxyacetic acid ester, 3-[di(2-hydroxy-ethyl)amino]aniline, 4-amino-2-di[(2-hydroxyethyl)amino]-1-ethoxy-benzene, 5-methyl-2-(1-methylethyl)phenol, 3-[(2-hydroxyethyl)amino]aniline, 3-[(2-aminoethyl)amino]aniline, 1,3-di(2,4-diaminophenoxy)propane, di(2,4-diaminophenoxy)methane, 1,3-diamino-2,4-dimethoxybenzene, 2,6-bis(2-hydroxyethyl)aminotoluene, 4-hydroxyindole, 3-dimethylaminophenol, 3-diethylaminophenol, 5-amino-2-methylphenol, 5-amino-4-fluoro-2-methyl-phenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methyl-phenol, 3-amino-2-chloro-6-methylphenol, 3-aminophenol, 2-[(3-hydroxyphenyl)-amino]acetamide, 5-[(2-hydroxyethyl)amino]-4-methoxy-2-methylphenol, 5-[(2-hydroxyethyl)amino]-2-methylphenol, 3-[(2-hydroxyethyl)amino]-phenol, 3-[(2-methoxyethyl)amino]-phenol, 5-amino-2-ethyl-phenol, 5-amino-2-methoxy-phenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxy-ethyl)amino]-2-methylphenol, 2-amino-3-hydroxypyridine, 5-amino-4-chloro-2-methylphenol, 1-naphthol, 2-methyl-1-naphthol, 1,5-dihydroxynaphthalene, 1,7-dihydroxy-naphthalene, 2,3-dihydroxynaphthalene, 2,7-dihydroxy-naphthalene, 2-methyl-1-naphthol-acetate, 1,3-dihydroxybenzene, 1-chloro-2,4-dihydroxy-benzene, 2-chloro-1,3-dihydroxybenzene, 1,2-dichloro-3,5-dihydroxy-4-methylbenzene, 1,5-dichloro-2,4-dihydroxy-benzene, 1,3-dihydroxy-2-methyl-benzene, 3,4-methylendioxy-phenol, 3,4-methylendioxy-aniline, 5-[(2-hydroxy-ethyl)amino]-1,3-benzodioxol, 6-bromo-1-hydroxy-3,4-methylendioxybenzene, 3,4-diaminobenzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-dihydroxyindole, 5,6-dihydroxyindolene, 5-hydroxyindole, 6-hydroxyindole, 7-hydroxyindole and 2,3-indolendione. The suitable coupler compounds can be used individually, or together with each other in a mixture.

Suitable self-coupling oxidation dye compounds include 2-amino-5-methylphenol, 2-amino-6-methylphenol, 2-amino-5-ethoxyphenol and 2-propyl-amino-5-aminopyridine.

The total amount of oxidation dye precursor compounds in the oxidation dye composition of the invention amounts to from about 0.01 to 12 percent by weight, preferably from about 0.2 to 6 percent by weight.

To obtain various color shades or tones the compositions according to the invention can also contain conventional direct-dyeing dye compounds, for example triphenylmethane dyestuffs, aromatic nitro dyestuffs, azo dyestuffs, quinoline dyestuffs, cationic dyestuffs or anionic dyestuffs. These dyestuffs include, for example, 1,4-bis[(2-hydroxyethyl)amino]-2-nitrobenzene, 1-(2-hydroxyethyl)-amino-2-nitro-4-[di(2-hydroxyethyl)-amino]benzene (HC Blue No. 2), 1-amino-3-methyl-4-[(2-hydroxyethyl)-amino]-6-nitrobenzene (HC Violet No. 1), 4-[ethyl-(2-hydroxyethyl)amino]-1-[(2-hydroxyethyl)amino]-2-nitro-benzene-hydrochloride (HC Blue No. 12), 4-[di(2-hydroxyethyl)amino]-1-[(2-methoxy-ethyl)amino]-2-nitrobenzene (HC Blue No. 11), 1-[(2,3-dihydroxypropyl)amino]-4-[methyl-(2-hydroxyethyl)-amino]-2-nitrobenzene (HC Blue No. 10), 1-[(2,3-dihydroxypropyl)-amino]-4-[ethyl-(2-hydroxyethyl)amino]-2-nitrobenzene hydrochloride (HC Blue No. 9), 1-(3-hydroxypropylamino)4-[di(2-hydroxy-ethyl)amino]-2-nitrobenzene (HC Violet No. 2), 1-methylamino-4-[methyl-(2,3-dihydroxypropyl)amino]-2-nitrobenzene (HC Blue No. 6), 2-((4-amino-2-nitro-phenyl)amino)-5-dimethylaminobenzoic acid (HC Blue No. 13), 1-amino-4-[(2-hydroxyethyl)amino]-2-nitrobenzene (HC Red No. 7), 2-amino-4,6-dinitrophenol, 4-amino-2-nitro-diphenylamine (HC Red No. 1), 1-amino-4-[di(2-hydroxyethyl)-amino]-2-nitrobenzene hydrochloride (HC Red No. 13), 1-amino-5-chloro-4-[(2-hydroxyethyl)amino]-2-nitrobenzene, 4-amino-1-[(2-hydroxyethyl)amino]-2-nitro-benzene (HC Red No. 3), 4-amino-3-nitrophenol, 4-[(2-hydroxyethyl)amino]-3-nitrophenol, 1-[(2-aminoethyl)amino]-4-(2-hydroxyethoxy)-2-nitrobenzene (HC Orange No. 2), 4-(2,3-dihydroxypropoxy)-1-[(2-hydroxyethyl)amino]-2-nitro-benzene (HC Orange No. 3), 1-amino-5-chloro-4-[(2,3-dihydroxypropyl)amino]-2-nitrobenzene (HC Red No. 10), 5-chloro-1,4-[di(2,3-dihydroxypropyl)amino]-2-nitrobenzene (HC Red No. 11), 2-[(2-hydroxyethyl)amino]-4,6-dinitrophenol, 4-ethylamino-3-nitrobenzoic acid, 2-[(4-amino-2-nitrophenyl)amino]benzoic acid, 2-chloro-6-ethylamino-4-nitrophenol, 2-amino-6-chloro-4-nitrophenol, 4-[(3-hydroxypropyl)-amino]-3-nitrophenol, 2,5-diamino-6-nitropyridine, 1,2,3,4-tetrahydro-6-nitroquinoxaline, 7-amino-3,4-dihydro-6-nitro-2H-1,4-benzoxazine (HC Red No. 14), 1-amino-2-[(2-hydroxyethyl)amino]-5-nitrobenzene (HC Yellow No. 5), 1-(2-hydroxyethoxy)-2-[(2-hydroxyethyl)amino]-5-nitrobenzene (HC Yellow No. 4), 1-[(2-hydroxyethyl)amino-2-nitrobenzene (HC Yellow No. 2), 2-[(2-hydroxy-ethyl)-amino]-1-methoxy-5-nitrobenzene, 2-amino-3-nitrophenol, 1-(2-hydroxy-ethoxy)-3-methylamino-4-nitrobenzene, 2,3-(dihydroxypropoxy)-3-methyl-amino-4-nitrobenzene, 2-[(2-hydroxyethyl)amino]-5-nitrophenol (HC Yellow No. 11), 3-[(2-aminoethyl)-amino]-1-methoxy-4-nitrobenzene hydrochloride (HC Yellow No.9), 1-[(2-ureidoethyl)amino]-4-nitrobenzene, 4-[(2,3-dihydroxypropyl)amino]-3-nitro-1-trifluoromethylbenzene (HC Yellow No. 6), 1-chloro-2,4-bis[(2-hydroxyethyl)amino]-5-nitrobenzene (HC Yellow No. 10), 4-[(2-hydroxyethyl)amino]-3-nitro-1-methylbenzene,1-chloro-4-[(2-hydroxyethyl)-amino]-3-nitrobenzene (HC Yellow No. 12), 4-[(2-hydroxyethyl)-amino]-3-nitro-1-trifluoromethylbenzene (HC Yellow No. 13), 4-[(2-hydroxyethyl)amino]-3-nitro-benzonitrile (HC Yellow No. 14), 4-[(2-hydroxyethyl)amino]-3-nitrobenzamide (HC Yellow No. 15),1,4-di[(2,3-dihydroxypropyl)amino]-9,10-anthraquinone, 1-[(2-hydroxyethyl)amino]-4-methylamino-9,10-anthraquinone (C.I. 61505, Disperse Blue No. 3), 2-[(2-aminoethyl)amino]-9,10-anthraquinone (HC Orange No. 5), 1-hydroxy-4-[(4-methyl-2-sulfophenyl)amino]-9,10-anthraquinone, 1-[(3-amino-propyl)amino]-4-methyl-amino-9,10-anthraquinone (HC Blue No. 8),1-[(3-amino-propyl)-amino]-9,10-anthraquinone (HC Red No. 8),1,4-diamino-2-methoxy-9,10-anthraquinone (C.I. 62015, Disperse Red No. 11, Solvent Violet No. 26), 1,4-dihydroxy-5,8-bis[(2-hydroxyethyl)amino]-9,10-anthraquinone (C.I. 62500, Disperse Blue No. 7, Solvent Blue No. 69), 9-(dimethylamino)-benzo[a]-phenoxazin-7-ium chloride (C.I. 51175; Basic Blue No. 6), di[4-(diethylamino)-phenyl][4-(ethylamino)naphthyl]-carbenium chloride (C.I. 42595; Basic Blue No. 7), 3,7-di(dimethylamino)-phenothiazin-5-ium chloride (C.I. 52015; Basic Blue No. 9), di[4-(dimethylamino)phenyl][4-(phenylamino)-naphthyl]-carbenium chloride (C.I. 44045; Basic Blue No. 26), 2-[(4-(ethyl(2-hydroxyethyl)amino)-phenyl)azo]-6-methoxy-3-methylbenzothiazolium methylsulfate (C.I. 11154; Basic Blue No.41), 8-amino-2-bromo-5-hydroxy-4-imino-6-[(3-(trimethylammonio)-phenyl)-amino]-1(4H)-naphthalenone chloride (C.I. 56059; Basic Blue No. 99), bis[4-(dimethylamino)-phenyl][4-(methylamino)phenyl]carbenium chloride (C.I. 42535; Basic Violet No. 1), tris[4-(dimethylamino)phenyl]carbenium chloride (C.I. 42555; Basic Violet No. 3), 2-[3,6-(diethylamino)-dibenzopyranium-9-yl-benzoic acid chloride (C.I. 45170; Basic Violet No. 10), di(4-aminophenyl)(4-amino-3-methylphenyl)-carbenium chloride (C.I. 42510; Basic Violet No. 14), 1,3-bis[(2,4-diamino-5-methylphenyl)azo]-3-methylbenzene (C.I. 21010; Basic Brown No. 4), 1-[(4-aminophenyl)azo]-7-(trimethylammonio)-2-naphthol chloride (C.I. 12250; Basic Brown No. 16),1-[(4-amino-2-nitrophenyl)-azo-7-(trimethylammonio)-2-naphthol-chloride (Basic Brown No. 17),1-[(4-amino-3-nitrophenyl)azo]-7-(trimethylammonio)-2-naphthol chloride (C.I. 12251; Basic Brown No. 17), 3,7-diamino-2,8-dimethyl-5-phenylphenazinium chloride (C.I. 50240; Basic Red No. 2),1,4-dimethyl-5-[(4-(dimethylamino)phenyl)-azo]-1,2,4-triazolium chloride (C.I. 11055; Basic Red No. 22), 2-hydroxy-1-[(2-methoxy-phenyl)azo]-7-(trimethyl-ammonio)-naphthalene chloride (C.I. 12245; Basic Red No. 76), 2-[2-((2,4-dimethoxyphenyl)amino)ethenyl]-1,3,3-trimethyl-3H-indol-1-ium chloride (C.I. 48055; Basic Yellow No. 11), 3-methyl-1-phenyl-4-[(3-(trimethylammonio)-phenyl)azo]-pyrazol-5-one chloride (C.I 12719; Basic Yellow No. 57), bis[4-diethylamino)phenyl]-phenyl carbenium hydrogen sulfate (1:1) (C.I. 42040; Basic Green No. 1), 1-[di(2-hydroxyethyl)-amino]-3-methyl-4-[(4-nitrophenyl)azo]-benzene (C.I. 11210, Disperse Red No. 17), 4-[(4-amino-phenyl)-azo]-1-[di(2-hydroxyethyl)-amino]-3-methylbenzene (HC Yellow No. 7), 2,6-diamino-3-[(pyridine-3-yl)azo]-pyridine, 6-hydroxy-5-[(4-sulfophenyl)-azo]-2-naphthalene sulfonic acid, disodium salt (C.I. 15985; Food Yellow No. 3; FD&C Yellow No. 6), 2,4-dinitro-1-naphthol-7-sulfonic acid, disodium salt (C.I. 10316; Acid Yellow No. 1; Food Yellow No. 1), 2-(indane-1,3-dion-2-yl)quinolin-x,x-sulfonic acid (mixture of mono- and disulfonic acid) (C.I. 47005;D&C Yellow No. 10; Food Yellow No. 13; Acid Yellow No. 3), 5-hydroxy-1-(4-sulfophenyl)-4-[(4-sulfophenyl)-azo]pyrazol-3-carbonic acid, trisodium salt (C.I. 19140; Food Yellow No. 4; Acid Yellow No. 23), 9-(2-carboxyphenyl)-6-hydroxy-3H-xanthen-3-one (C.I. 45350; Acid Yellow No. 73; D&C Yellow No. 8), 5-[(2,4-dinitrophenyl)-amino]-2-phenyl-amino-benzene sulfonic acid, sodium salt (C.I. 10385; Acid Orange No. 3), 4-[(2,4-dihydroxy-phenyl)azo]-benzosulfonic acid, monosodium salt (C.I. 14270; Acid Orange No. 6), 4-[(2-hydroxynaphth-1-yl)azo]-benzene sulfonic acid, sodium salt (C.I. 15510; Acid Orange No. 7), 4-[(2,4-dihydroxy-3-[(2,4-dimethylphenyl)-azo]phenyl)azo]benzene sulfonic acid, sodium salt (C.I. 20170; Acid Orange No. 24), 4-hydroxy-3-[(4-sulfonaphth-1-yl)azo]-1-naphthalene sulfonic acid, disodium salt (C.I. 14720; Acid Red No. 14), 6-hydroxy-5-[(4-sulfonaphth-1-yl)azo]-2,4-naphthalene-disulfonic acid, trisodium salt (C.I. 16255; Ponceau 4R; Acid Red No. 18), 3-hydroxy-4-[(4-sulfonaphth-1-yl)azo]-2,7-naphthalene disulfonic acid, trisodium salt (C.I. 16185; Acid Red No.27), 8-amino-11-hydroxy-2-(phenylazo)-3,6-naphthalene disulfonic acid, disodium salt (C.I 17200; Acid Red No. 33), 5-(acetylamino)-4-hydroxy-3-[(2-methylphenyl)azo]-2,7-naphthalene disulfonic acid, disodium salt (C.I. 18065; Acid Red No. 35), 2-(3-hydroxy-2,4,5,7-tetraiododibenzopyran-6-one-9-yl)-benzoic acid, disodium salt (C.I. 45430; Acid Red No. 51), N-[6-(diethylamino)-9-(2,4-disulfophenyl)-3H-xanthen-3-yliden]-N-ethylethane ammonium hydroxide, inner salt, sodium salt (C.I. 45100; Acid Red No. 52), 8-[(4-(phenylazo)-phenyl)azo]-7-napthol-1,3-disulfonic acid, disodium salt (C.I. 27290; Acid Red No. 73), 2′, 4′, 5′, 7′-Tetrabromo-3′, 6′-dihydroxyspiro-[isobenzofuran-1(3H),9′-[9H]xanthen]-3-one-disodium salt (C.I. 45380; Acid Red No. 87), 2′, 4′, 5′, 7′-tetrabromo-4,5,6,7-tetrachloro-3′, 6′-dihydroxyspiro-[isobenzofuran-1(3H), 9′[9H]xanthen]-3-one, disodium salt (C.I. 45410; Acid Red No. 92), 3′, 6′-dihydroxy-4′, 5′-diiodospiro[isobenzofuran-1(3H),9′(9H)-xanthen]-3-one, disodium salt (C.I. 45425; Acid Red No. 95), (2-sulfophenyl)di[4-(ethyl((4-sulfophenyl)-methyl)amino)phenyl]carbenium, disodium salt, betaine (C.I. 42090; Acid Blue No. 9; FD&C Blue No. 1), 1,4-bis[(2-sulfo-4-methylphenyl)amino]-9,10-anthraquinone, disodium salt (C.I. 61570; Acid Green No. 25), bis[4-(dimethyl-amino)phenyl]-(3,7-disulfo-2-hydroxynaphth-1-yl)carbenium, inner salt, monosodium salt (C.I. 44090; Food Green No. 4; Acid Green No. 50), bis[4-(diethylamino)phenyl](2,4-disulfophenyl)carbenium, inner salt, sodium salt (2:1) (C.I. 42045; Food Blue No. 3; Acid Blue No. 1), bis[4-(diethylamino)-phenyl](5-hydroxy-2,4-disulfophenyl)carbenium, inner salt, calcium salt (2:1) (C.I. 42051; Acid Blue No. 3), 1-amino-4-(cyclohexylamino)-9,10-anthraquinone-2-sulfonic acid sodium salt (C.I. 62045; Acid Blue No. 62), 2-(1,3-Dihydro-3-oxo-5-sulfo-2H-indol-2-yliden)-2,3-dihydro-3-oxo-1H-indol-5-sulfonic acid, disodium salt (C.I. 73015; Acid Blue No. 74), 9-(2-carboxy-phenyl)-3-[(2-methylphenyl)-amino]-6-[(2-methyl-4-sulfophenyl)amino]xanthylium inner salt, monosodium salt (C.I. 45190; Acid Violet No. 9), 1-hydroxy-4-[(4-methyl-2-sulfophenyl)amino]-9,10-anthraquinone, sodium salt (C.I. 60730; D&C Violet No. 2; Acid Violet No. 43), bis[3-nitro-4-[(4-phenylamino)-3-sulfophenyl-amino]-phenyl]sulfone (C.I. 10410; Acid Brown No. 13), 5-amino-4-hydroxy-6-[(4-nitrophenyl)azo]-3-(phenylazo)-2,7-naphthalene-disulfonic acid , disodium salt (C.I. 20470; Acid Black No. 1), 3-hydroxy-4-[(2-hydroxynaphth-1-yl)azo]-7-nitro-1-naphthalene-sulfonic acid chromium complex (3:2) (C.I. 15711; Acid Black No. 52), 3-[(2,4-dimethyl-5-sulfophenyl)azo]-4-hydroxy-1-naphthalene-sulfonic acid, disodium salt (C.I. 14700; Food Red No. 1; FD&C Red No. 4), 4-(acetylamino)-5-hydroxy-6-[(7-sulfo-4-[(4-sulfophenyl)azo]naphth-1-yl)azo]-1,7-naphthalene disulfonic acid, tetrasodium salt (C.I. 28440; Food Black No. 1) and 3-hydroxy-4-(3-methyl-5-oxo-1-phenyl-4,5-dihydro-1H-pyrazol-4-yl-azo)-naphthalen-1-sulfonic acid, sodium salt, chromium complex (Acid Red No. 195). These latter compounds can be used alone or in mixtures with each other in the compositions of the invention.

The total amount of direct-dyeing dye compounds in the composition according to the invention amounts to about 0.01 to 7 percent by weight, preferably about 0.2 to 4 percent by weight.

E. Sagarin, in “Cosmetics, Science and Technology”, Interscience Publishers Inc., New York (1957), pp. 503 and following and H. Janistyn, “Hand-buch der Kosmetika und Riechstoffe[Handbook of Cosmetics and Perfumes]”, Volume 3 91973), pp. 388 and following and K. Schrader in “Gundlagen und Rezepturen der Kosmetika[Fundamentals and Formulations of Cosmetics]”, 2^{nd} Edition (1989), pp. 782 to 815, among others, describe additional dye known and conventional compounds for hair dyeing, which can also be contained in the dye compositions according to the invention.

Although oxidation dye compositions are preferred, it is understandably possible that the dye composition according to the invention is in the form of a non-oxidative dye composition based on the above-described direct-dyeing dye compounds.

Furthermore antioxidants, such as ascorbic acid, thioglycolic acid or sodium sulfite, as well as complex formers for heavy metals, such as ethylenediaminetetraacetic acid or nitriloacetic acid, can be present in the composition according to the invention in amounts of up to about 0.5 percent by weight. Perfume oils can also be contained in the dye composition according to the invention in an amount of up to about 1 percent by weight. Understandably the above-described hair dye composition can contain other additives, as needed, for hair dye composition, for example preservatives and perfume oils, antioxidants, for example sodium sulfite, thioglycolic acid or ascorbic acid; complex formers; solvents, such as water, lower aliphatic alcohols, for example ethanol, propanol and isopropanol, or glycols, such as glycerol and 1,2-propylene glycol; wetting agents or emulsifiers selected from the classes of anionic, cationic, amphoteric or nonionic surface-active substances; thickeners, such as higher fatty alcohols, starches or cellulose derivative compounds; softeners; petrolatum (Vaseline®); silicone oils, paraffin oils and fatty acids, as well as care materials, such as cationic resins, lanolin derivative compounds, cholesterol, vitamins, pantothenic acids and betaines. The above-mentioned ingredients are used in the usual conventional amounts suitable for their purposes. For example the wetting agents and emulsifiers are present in concentrations of from 0.1 to 30 percent by weight, the thickeners, in amounts of from 0.1 to 25 percent by weight and the care materials in concentrations of from 0.1 to 5.0 percent by weight. The addition of nonionic and/or anionic surfactants or emulsifiers, such as fatty alcohol sulfates, especially lauryl sulfates and sodium cocoyl sulfates, ethoxylated fatty alcohol sulfates, especially sodium lauryl ether sulfates with 2 to 4 ethylene oxide units per molecule, ethoxylated fatty acid esters, ethoxylated nonyl phenols, ethoxylated fatty alcohols, alkyl benzene sulfonates or fatty acid alkanol amides, in total amounts of about 0.1 to 30 percent by weight, preferably from 0.2 to 15 percent by weight, is particularly preferred.

The pH values of the non-oxidative dye compositions according to the invention based on direct-dyeing dye compounds are in a pH range of about 5 to 10, preferably 6 to 9. The pH values of the oxidative dye compositions according to the invention are in a pH range of about 6 to 12, preferably 9 to 11, while the pH values of the ready-to-apply oxidation dye mixture (which means the mixture of the dye composition according to the invention with an oxidizing agent) amounts to from about 5.5 to 10, preferably 6 to 9.

The pH is adjusted according to the composition and desired pH value of the dye composition by addition of ammonia or organic amines, such as glucamines, aminomethylpropanol, monoethanolamine or triethanolamine, inorganic bases, for example sodium hydroxide, potassium hydroxide, sodium carbonates or calcium carbonates, or by addition or organic or inorganic acids, such as lactic acid, citric acid, acetic acid or phosphoric acid.

The composition according to the invention is preferably packaged in the form of an aqueous or aqueous-alcoholic preparation, for example as a thickened solution, as an emulsion, as a cream or as a gel. Lower alcohols with one to four carbon atoms, especially ethanol and isopropanol, can be used as the alcohols suitable for cosmetic purposes.

For oxidative dyeing the above-described dye composition are mixed with an oxidizing agent or composition (which, for example, may be thickened with a nonionic amphiphilic associative thickener) immediately prior to application. Then the resulting dyeing mixture is applied to the fibers in an amount sufficient for the dyeing of the fibers, usually from about 60 to 200 grams.

In as much as the dye composition according to the invention does not contain oxidation dye pre-cursor compounds or does contain oxidation dye pre-cursor compounds that are easily oxidized with air oxygen, that dye composition can be directly applied to the keratin fibers without previous mixing with an oxidizing agent.

Hydrogen peroxide, or its addition products with urea, melamine or sodium borate, in the form of a 1 to 12 percent, preferably 1.5 to 6 percent, aqueous solution, can be used as the oxidizing agent for development of the dyed fiber color. The mixture ratio of dye composition to oxidizing agent is usually about 5:1 to 1:2, preferably 1:1. The content of the oxidizing agent in the ready-to-apply preparation preferably amounts about 0.5 to 8 percent by weight, especially from 1 to 4 percent by weight.

The ready-to-apply dyeing mixture or dye composition is allowed to act on the keratin fibers (for example, human hair) for about 10 to 45 minutes, preferably about 15 to 30 minutes, at 15 to 50° C., then the mixture or composition is rinsed from the fibers with water and the dyed fibers are dried. If necessary the dyed fibers can be washed with a shampoo and even with a weak organic acid, such as tartaric acid. Subsequently the keratin fibers are dried.

It has been found that the viscosity of the oxidation dye compositions according to the invention can be easily increased according to the desire of the user without more. In this way a simpler and more economical basic formulations are possible. The dye compositions prepared according to the invention fulfill the requirements in regard to viscosity adjustment, application properties and adherence in better ways, have improved flow behavior during application and is easy to apply.

The following examples should illustrate the invention in more detail, but without limiting the appended claims.

### EXAMPLES

### Example 1

Dye Thickener for Subsequent Adjustment of the Viscosity of the Dye Composition

| | | |
|---|---|---|
| | 30.0 g | PEG-180/Laureth-50/TMMG Copolymer, 20% aqueous |
| | | solution (Pure Thix ®M) |
| | 0.2 g | PHB methyl ester |
| | 1.8 g | propylene glycol |
| to | 100.0 g | water, desalinated |

### Example 2

Dye Thickener for Subsequent Adjustment of the Viscosity of the Dye Composition with Additional Care Material

| | | |
|---|---|---|
| | 1.3 g | Polyether-1,20 percent aqueous solution (Pure Thix ®HH) |
| | 0.2 g | PHB methyl ester |
| | 1.8 g | propylene glycol |
| | 0.6 g | dimethicone propyl PG betaine |
| | 1.0 g | dimethicone copolyol |
| to | 100.0 g | water, desalinated |

### Example 3

Oxidation Hair Dye Composition

| Component (A): | | Dye Carrier |
|---|---|---|
| | 2.0 g | PEG-180/Laureth-50/TMMG Copolymer, |
| | | (Pure Thix ® TX-1450) |
| | 3.0 g | sodium lauryl alcohol diglycol ether sulfate, 28 percent |
| | | aqueous solution |
| | 2.8 g | 2,5-diaminotoluene sulfate |
| | 1.0 g | resorcinol |
| | 0.4 g | m-aminophenol |
| | 0.2 g | 2-amino-4-(2′-hydroxyethylamino)anisole sulfate |
| | 0.3 g | ascorbic acid |
| | 0.1 g | ethylenediaminetetraacetic acid |
| | 12.2 g | ammonia, 25% aqueous solution |
| | 2.0 g | ethanol |
| to | 100.0 g | water |

| Component (B): | | Hydrogen Peroxide Emulsion |
|---|---|---|
| | 10.0 g | cetyl stearyl alcohol |
| | 1.5 g | cholesterol |
| | 4.0 g | sodium lauryl alcohol diglycol ether sulfate, 28% |
| | | aqueous solution |
| | 17.0 g | hydrogen peroxide, 35% aqueous solution |
| | 0.3 g | perfume |
| to | 100.0 g | water |

Immediately prior to application 40 g of the liquid dye carrier (A) is mixed with 80 g of the hydrogen peroxide emulsion (B), in a mixture ratio of (A):(B) of 1:2 and 120 g of this mixture is applied to gray human hair. After an acting time of 20 minutes at room temperature the hair is rinsed with water and subsequently dried. The hair treated in this way has a uniform dark brown color. The composition adheres very satisfactorily to the hair without running off.

### Example 4

Gel-form Oxidation Hair Dye Composition for Bright Dyeing

| Component (A): | | Dye Carrier |
|---|---|---|
| | 3.00 g | PEG-180/Octoxynol-40/TMMG Copolymer |
| | 6.00 g | nonylphenol ethoxylated with 4 mol ethylene oxide |
| | 6.00 g | oleic acid |
| | 0.50 g | para-phenylenediamine |
| | 0.07 g | resorcinol |
| | 5.00 g | sodium lauryl alcohol diglycol ether sulfate, 28 |
| | | percent aqueous solution |
| | 1.00 g | ethylenediaminetetraacetic acid, disodium salt |
| | 18.00 g | ammonia, 25% aqueous solution |
| | 8.00 g | ethanol |
| to | 100.0 g | water |

| Component (B): | | Hydrogen Peroxide Emulsion |
|---|---|---|
| | 10.0 g | cetyl stearyl alcohol |
| | 1.5 g | cholesterol |
| | 4.0 g | sodium lauryl alcohol diglycol ether sulfate, 28% |
| | | aqueous solution |
| | 35.0 g | hydrogen peroxide, 35% aqueous solution |
| | 0.3 g | perfume |
| to | 100.0 g | water |

Immediately prior to application 40 g of the liquid dye carrier (A) is mixed with 80 g of the hydrogen peroxide emulsion (B), in a mixture ratio of (A):(B) of 1:2 and 120 g of this mixture is applied to gray human hair. After an acting time of 20 minutes at room temperature the hair is rinsed with water and subsequently dried. The hair treated in this way has a bright brown color uniformly from the tips to the roots. The composition adheres very satisfactorily to the hair without running off.

### Example 5

Cream-form Oxidation Hair Dye Composition for Bright Dyeing

| | | |
|---|---|---|
| | 3.00 g | PEG-180/Laureth-50/TMMG Copolymer, |
| | | (Pure Thix ® TX-1450) |
| | 15.00 g | cetyl alcohol |
| | 3.50 g | sodium lauryl alcohol diglycol ether sulfate, 28 percent |
| | | aqueous solution |
| | 3.00 g | monoethanolamine |
| | 1.30 g | 1-methyl-2,5-diaminobenzene |
| | 1.00 g | bees wax |
| | 0.65 g | resorcinol |
| | 0.50 g | keratin hydrolyzate |
| | 0.50 g | silk protein hydrolyzate |
| | 0.50 g | 2-amino-6-chloro-4-nitrophenol |
| | 0.30 g | ascorbic acid |
| to | 100.0 g | water |

50 g of the above-described hair dye composition are mixed with 50 g of 2 percent aqueous hydrogen peroxide solution immediately prior to application. The mixture so obtained is subsequently applied to bleached natural hair. After an acting time of 30 minutes at 40° C. the hair is rinsed with water and dried. A uniform luminous full gold-orange dyed hair color is obtained.

### Example 6

Non-oxidative Hair Dye Composition

| | | |
|---|---|---|
| | 1.8 g | PEG-180/Laureth-50/TMMG Copolymer, |
| | | (Pure Thix ® TX-1450) |
| | 5.0 g | sodium lauryl sulfate |
| | 1.5 g | 2-amino-6-chloro-4-nitrophenol |
| | 1.0 g | monoethanolamine |
| | 1.0 g | bees wax |
| | 0.5 g | keratin hydrolyzate |
| | 0.3 g | silk protein hydrolyzate |
| | 0.2 g | glycine |
| to | 100.0 g | water |

The hair dye composition is applied uniformly on the hair. Because of the outstanding viscosity properties the composition adheres very well to the hair. After an acting time of 20 minutes at 20° C. the hair is rinsed with lukewarm water, put in a hairstyle or hair do and dried. The hair so treated has a uniform luminous or shinning intense gold-orange color.

All percentages, unless otherwise indicated, are percentages by weight.

The disclosure in German Patent Application 100 23 028.8 of May 11, 2000 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in compositions and methods for dyeing keratin fibers, especially human hair, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A composition for dyeing keratin fibers, said composition comprising
at least one dye compound selected from the group consisting of oxidation dye pre-cursor compounds and direct-dyeing dye compounds;
at least one nonionic amphiphilic associative thickener; and
a cosmetic carrier.

2. The composition as defined in claim 1, wherein said at least one nonionic amphiphilic associative thickener is selected from the group consisting of hydrophobically modified polyalkylene glycols.

3. The composition as defined in claim 1, wherein said at least one nonionic amphiphilic associative thickener is selected from the group consisting of hydrophobically modified aminoplast polyether copolymers.

4. The composition as defined in claim 1, wherein said at least one nonionic amphiphilic associative thickener is selected from the group consisting of polymer of formula (I): 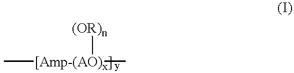
wherein Amp represents an aminoplast monomer, an aminoplast oligomer group or an aminoplast polymer group; AO represents an alkylene oxide group, R represents hydrogen, a C₁- to C₄-alkyl group or a C₁- to C₄-acyl group and x and y are greater than 1.

5. The composition as defined in claim 1, wherein said at least one nonionic amphiphilic associative thickener is selected from the group consisting of reaction products of acid catalyzed polycondensation of a glycoluril of the formula (II), 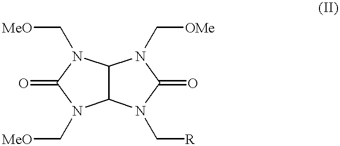
wherein R represents H or OMe, with polyethylene oxide diols having an ethoxylation degree of 20 to 500, and, if necessary, with an if need be ethoxylated hydrophobic alcohol or an alkyphenol, a thiol, a carboxamide, a carbamate or a hydrophobic carboxylic acid.

6. The composition as defined in claim 5, wherein said glycoluril of the formula (II) is 1,3,4,6-tetramethoxymethyl glycoluril.

7. The composition as defined in claim 1, wherein said at least one nonionic amphiphilic associative thickener is selected from the group consisting of polyether-1, PEG-180/octoxynol-40/TMMG copolymer and PEG-180/laureth-50/TMMG copolymer.

8. The composition as defined in claim 1, containing from 0.01 to 12 percent by weight of a total amount of said oxidation dye pre-cursor compounds.

9. The composition as defined in claim 1, wherein said dye carrier comprises at least one solvent ingredient selected from the group consisting of water, glycols and alcohols having from 1 to 4 carbon atoms; from 0.1 to 5.0 percent by weight of at least one care material and from 0.1 to 30 percent by weight of at least one surfactant member selected from the group consisting of anionic surfactants, cationic surfactants, amphoteric surfactants and nonionic surfactants.

10. A ready-to-apply dyeing mixture made by mixing an oxidizing agent-containing composition with a dye composition in a weight ratio of the dye composition to the oxidizing agent-containing composition of 5:1 to 1:2;
wherein said dye composition contains from 0.01 to 12 percent by weight of a total amount of oxidation dye pre-cursor compounds and at least one nonionic amphiphilic associative thickener.

11. The ready-to-apply dyeing mixture as defined in claim 10, wherein said at least one nonionic amphiphilic associative thickener is selected from the group consisting of hydrophobically modified polyalkylene glycols and hydrophobically modified aminoplast polyether copolymers.

12. The ready-to-apply dyeing mixture as defined in claim 10, having a pH of 5.5 to 10 and wherein said dye composition includes at least one direct-dyeing dye compound.

13. A method of dyeing keratin fibers, said method comprising the steps of:
a) mixing an oxidizing agent-containing composition with a dye composition in a weight ratio of the dye composition to the oxidizing agent-containing composition of 5:1 to 1:2 to form a ready-to-apply dyeing mixture;
b) applying a sufficient amount of the ready-to-apply dyeing mixture to the keratin fibers in order to dye the keratin fibers;
c) after the applying step b), allowing the ready-to-apply dyeing mixture to act on the keratin fibers for an acting time of from 10 to 45 minutes at 15 to 50° C.; and
d) rinsing the fibers with water and then drying the fibers so treated;
wherein the dye composition contains from 0.01 to 12 percent by weight of a total amount of oxidation dye pre-cursor compounds and at least one nonionic amphiphilic associative thickener.

14. The method as defined in claim 13, wherein said at least one nonionic amphiphilic associative thickener is selected from the group consisting of hydrophobically modified polyalkylene glycols and hydrophobically modified aminoplast polyether copolymers.

15. The method as defined in claim 13, wherein said oxidizing agent-containing composition comprises hydrogen peroxide or an addition product of hydrogen peroxide with urea, melamine or sodium borate.

16. A method of dyeing keratin fibers, said method comprising the steps of:
a) applying a sufficient amount of a dye composition to the keratin fibers without previous mixing with an oxidizing agent, in order to dye the keratin fibers;
b) after the applying of step a), allowing the dye composition to act on the keratin fibers; and
c) after the allowing of step b), rinsing the keratin fibers with water and then drying the keratin fibers so treated;
wherein the dye composition contains from 0.01 to 7 percent by weight of at least one direct-dyeing dye compound and at least one nonionic amphiphilic associative thickener.

17. The method as defined in claim 16, wherein said at least one nonionic amphiphilic associative thickener is selected from the group consisting of hydrophobically modified polyalkylene glycols and hydrophobically modified aminoplast polyether copolymers.

18. The composition as defined in claim 16, wherein said at least one nonionic amphiphilic associative thickener is selected from the group consisting of polyether-1, PEG-1 80/octoxynol-40/TMMG copolymer and PEG-180/laureth-50/TMMG copolymer.

19. The method as defined in claim 16, wherein said at least one direct-dyeing dye compound is selected from the group consisting of trimethylene dye compounds, aromatic nitro dye compounds, azo dye compounds, quinoline dye compounds, cationic dye compounds and anionic dye compounds.
